(19) United States
(12) Patent Application Publication
(10) Pub. No.: US 2009/0263357 A1
(43) Pub. Date: Oct. 22, 2009

(54) Therapeutic Reprogramming, Hybrid Stem Cells and Maturation

(75) Inventors: Chauncey B. Sayre, Irvine, CA (US); Francisco J. Silva, Tusfin, CA (US)

Correspondence Address: K&L Gates LLP, 1900 MAIN STREET, SUITE 600, IRVINE, CA, 92614-7319, US

(73) Assignee: PrimeGen Biotech, LLC, Ivine, CA (US)

(21) Appl. No.: 12/013,282

(22) Filed: Jan. 11, 2008

Related U.S. Application Data
(63) Continuation of application No. 11/060,131, filed on Feb. 16, 2005, now abandoned.
Application No. 11/060,131 is a continuation-in-part of application No. 10/346,816, filed on Jan. 16, 2003, now abandoned.
Application No. 11/060,131 is a continuation-in-part of application No. 10/864,788, filed on Jun. 8, 2004, now abandoned.
(60) Provisional application No. 60/348,521, filed on Jan. 16, 2002.
(60) Provisional application No. 60/367,161, filed on Mar. 26, 2002.
(60) Provisional application No. 60/477,438, filed on Jun. 9, 2003.
(60) Provisional application No. 60/588,146, filed on Jul. 15, 2004.

(51) Int. Cl. A61K 35/12; C12N 5/06

(52) U.S. Cl. 424/93.7; 435/325

(57) ABSTRACT

Therapeutically programmed cells and methods for making such cells are provided. Therapeutically programmed cells are stem cells which have been matured such that they represent either a more differentiated state or a less differentiated state after contact with stimulatory factors. The therapeutically reprogrammed cells are suitable for cellular regenerative therapy and have the potential to differentiate into more committed cell lineages. Additionally, hybrid stem cells suitable for therapeutic reprogramming and cellular regenerative therapy are provided.

### RELATED APPLICATIONS

The present application is a continuation of U.S. patent application Ser. No. 11/060,131 filed Feb. 16, 2005 which is a continuation-in-part of U.S. patent application Ser. No. 10/346,816 filed Jan. 16, 2003, which claims priority to U.S. Provisional Patent Application No. 60/348,521 filed Jan. 16, 2002, and U.S. Provisional Patent Application No. 60/367,161 filed Mar. 26, 2002, and is a continuation-in-part of U.S. patent application Ser. No. 10/864,788 filed Jun. 8, 2004, which claims priority to U.S. Provisional Patent Application No. 60/477,438 filed Jun. 9, 2003, and claims priority to U.S. Provisional Patent Application No. 60/588,146 filed Jul. 15, 2004, the entire contents of which are incorporated herein by reference in their entirety.

### FIELD OF THE INVENTION

The present invention relates to the field of therapeutically reprogrammed cells. Specifically, therapeutically reprogrammed cells are provided that are not compromised by the aging process, are immunocompatible and will function in the appropriate post-natal cellular environment to yield functional cells after transplantation. Additionally, the present invention provides methods for providing hybrid stem cells suitable for therapeutic reprogramming, transplant and therapy.

### BACKGROUND OF THE INVENTION

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types (Kanatsu-Shinohara M. et al. Generation of pluripotent stem cells from neonatal mouse testis. Cell 119:1001-12, 2004).

Stem cells are a rare population of cells that can give rise to vast range of cells tissue types necessary for organ maintenance and function. These cells are defined as undifferentiated cells that have two fundamental characteristics; (i) they have the capacity of self-renewal, (ii) they also have the ability to differentiate into one or more specialized cell types with mature phenotypes. There are three main groups of stem cells; (i) adult or somatic (post-natal), which exist in all post-natal organisms, (ii) embryonic, which can be derived from a pre-embryonic or embryonic developmental stage and (iii) fetal stem cells (pre-natal), which can be isolated from the developing fetus. Each group of stem cells has their own advantages and disadvantages for cellular regeneration therapy, specifically in their differentiation potential and ability to engraft and function de novo in the appropriate or targeted cellular environment.

In the post-natal animal there are cells that are lineage-committed progenitor stem cells and lineage-uncommitted pluripotent stem cells, which reside in connective tissues providing the post-natal organism the cells required for continual organ or organ system maintenance and repair. These cells are termed somatic or adult stem cells and can be quiescent or non-quiescent. Typically adult stem cells share two characteristics: (i) they can make identical copies of themselves for long periods of time (long-term self renewal); and (ii) they can give rise to mature cell types that have characteristic morphologies and specialized functions.

Much of the understanding of stem cell biology has been derived from hematopoietic stem cells and their behavior after bone marrow transplantation. There are several types of adult stem cells within the bone marrow niche, each having unique properties and variable differentiation ability in relation to their cellular environment. Somatic stem cells isolated from human bone marrow transferred in utero into pre-immune sheep fetuses have the ability to xenograft into multiple tissues. Also within the bone marrow niche are mesenchymal stem cells, which have a wide range of non-hematopoietic differentiation abilities, including bone, cartilage, adipose, tendon, lung, muscle, marrow stroma, and brain tissues. In addition, neural stem cells, pancreatic, muscle, adipose, ovarian and spermatogonial stem cells have been found. The therapeutic utility of somatic or post-natal stem cells has been demonstrated and realized through the use of bone marrow transplants. However, adult somatic stem cells have genomes that have been altered by aging and cell division. Aging results in an accumulation of free radical insults, or oxidative damage, that can predispose the cell to forming neoplasms, reduce cell differentiation ability or induce apoptosis. Repeated cell division is directly related to telomere shortening which is the ultimate cellular clock that determines a cells functional life-span. Consequently, adult somatic stem cells have genomes that have sufficiently diverged from the physiological prime state found in embryonic and prenatal stem cells.

Unfortunately, virtually every somatic cell in the adult animal's body, including stem cells, possess a genome ravaged by time and repeated cell division. Thus until now the only means for obtaining stem cells having an undamaged, or prime state physiological genome, was to recover stem cells from aborted embryos or embryos formed using in vitro fertilization techniques. However, scientific and ethical considerations have slowed the progress of stem cell research using embryonic stem cells. Generation of embryonic stem cell lines had been thought to provide a renewable source of embryonic stem cells for both research and therapy but recent reports indicate that existing cell lines have been contaminated with immunogenic animal molecules (Martin M. et al., Human embryonic stem cells express an immunogenic nonhuman sialic acid. Nature Medicine 11:228-32, 2005).

Another problem associated with using adult stems cells is that these cells are not immunologically privileged, or can lose their immunological privilege after transplant. (The term “immunologically privileged” is used to denote a state where the recipient's immune system does not recognize the cells as foreign). Thus, only autologous transplants are possible in most cases when adult stem cells are used. Thus, most presently envisioned forms of stem cell therapy are essentially customized medical procedures and therefore economic factors associated with such procedures limit their wide ranging potential. Additional barriers to the use of currently available

Moreover, stem cells must be induced to mature into the organ or cell type desired to be useful as therapeutics. The factors affecting stem cell maturation in vivo are poorly understood and even less well understood ex vivo. Thus, present maturation technology relies on serendipity and biological processes largely beyond the control of the administering scientist or recipient.

Current research is focused on developing embryonic stem cells as a source of totipotent or pluripotent immunologically privileged cells for use in cellular regenerative therapy. However, since embryonic stem cells themselves may not be appropriate for direct transplant as they form teratomas after transplant, they are proposed as “universal donor” cells that can be differentiated into customized pluripotent, multipotent or committed cells that are appropriate for transplant. Additionally there are moral and ethical issues associated with the isolation of embryonic stem cells from human embryos.

Therefore, there is a need for sources of biologically useful, pluripotent stem cells having genomes in a nearly physiologically prime state. Furthermore, there is a need for sources of biologically useful, pluripotent stem cells having genomes in a nearly physiologically prime state that maintain their immunological privilege in recipients for a time period sufficient to be therapeutically useful. Additionally, there is a need to condition stem cell transplants either in vivo or ex vivo in order to maximize the potential that the transplanted stem cell will mature into the intended tissue.

### SUMMARY OF THE INVENTION

The present invention provides biologically useful pluripotent therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues.

In an embodiment of the present invention, a therapeutic reprogramming method is provided comprising isolating a stem cell, contacting the stem cell with a medium comprising stimulatory factors which induce development of the stem cell into a therapeutically reprogrammed cell, recovering the therapeutically reprogrammed cell from the medium and implanting the therapeutically reprogrammed cell, or a cell matured therefrom, into a host in need of a therapeutically reprogrammed cell. Stem cells suitable for therapeutic reprogramming according to the teachings of the present invention include embryonic stem cells, fetal stem cells, somatic stem cells, multipotent adult progenitor cells, hybrid stem cells, modified germ cells, adipose-derived stem cells and primordial sex cells. In one embodiment of the present invention the primordial sex cell is a spermatogonial stem cell.

In another embodiment of the present invention, stimulatory factors useful in the therapeutic reprogramming method of the present invention include chemicals, biochemicals, and cellular extracts. The chemical stimulating factors of the present invention are selected from the group consisting of 5-aza-2′-deoxycytidine, histone deacetylase inhibitor, n-butyric acid and trichostatin A. The cellular extract stimulatory factors of the present invention are selected from the group consisting of whole cell extracts, cytoplast extracts and karyoplast extracts. Cellular extracts useful in the therapeutic reprogramming methods of the present invention are isolated from stem cells including embryonic stem cells, fetal neural stem cells, multipotent adult progenitor cells, hybrid stem cells and primordial sex cells.

In an embodiment of the present invention the host in need of a therapeutically reprogrammed cell is a mammal, and more specifically a human. In another embodiment of the present invention the stem cells is isolated from the host in need of a therapeutically reprogrammed cell.

In yet another embodiment of the present invention, the therapeutic reprogramming method further includes the step of maturing said therapeutically reprogrammed cell to become committed to a tissue-specific lineage.

In an embodiment of the present invention, a therapeutic reprogramming method is provided comprising isolating a spermatogonial stem cell (SSC), contacting the SSC with a medium comprising stimulatory factors which induce development of the SSC into a totipotent cell, recovering the totipotent cell from the medium, and implanting the totipotent cell, or a cell matured therefrom, into a host in need of a therapeutically reprogrammed cell.

In another embodiment of the present invention, a therapeutic reprogramming method is provided comprising providing a hybrid stem cell, contacting the hybrid stem cell with a medium comprising stimulatory factors which induce development of the hybrid stem cells into a totipotent cell, recovering the totipotent cell from the medium; and implanting the totipotent cell, or a cell matured therefrom, into a host in need of a therapeutically reprogrammed cell.

In yet another embodiment of the present invention, a therapeutically reprogrammed cell is provided comprising an SSC which has been exposed to stimulatory factors which have caused the SSC to mature or differentiate into a totipotent or a pluripotent cell.

In an embodiment of the present invention, a therapeutically reprogrammed cell is provided comprising a pluripotent stem cell which has been exposed to stimulatory factors which have caused the pluripotent stem cell to mature or differentiate into a more committed cell lineage.

In another embodiment of the present invention, a method for making a hybrid stem cell is provided comprising obtaining a donor cell wherein the donor cell is diploid, obtaining a host cell, enucleating the host cell, fusing the donor cell, or nucleus thereof, and the host cell, and isolating the hybrid stem cell. Donor cells suitable for use in making hybrid stem cells according to the teachings of the present invention are selected from the group consisting of embryonic stem cells, somatic cells, primordial sex cells and therapeutically reprogrammed cells. In another embodiment of the present invention the donor cell is in G₀.

In yet another embodiment of the present invention, host cells suitable for use in making hybrid stem cells according to the method of the present invention are selected from the group consisting of embryonic stem cells, fetal neural stem cells and multipotent adult progenitor cells.

In an embodiment of the present invention, the method for making hybrid stem cells further comprises the step of culturing the host cell for four passages after the obtaining step and prior to the enucleating step.

In another embodiment of the present invention, the donor cell and the host cell suitable for making a hybrid stem cell are from a mammal. In yet another embodiment of the present invention, the donor cell and the host cell are from the same individual.

In an embodiment of the present invention, the host cell suitable for making a hybrid stem cell is enucleated by a process selected from the group consisting of chemical, mechanical, physical, x-ray irradiation and laser irradiation enucleation. In another embodiment of the present invention, the host cell is enucleated by cytochalasin D.

In yet another embodiment of the present invention the method of making a hybrid stem cell further comprises the step of culturing the enucleated host cell for approximately three days prior to fusing with the donor cell.

In an embodiment of the present invention, the fusing step of the method of making a hybrid stem cell comprises a fusion method selected from the group consisting of electrofusion, microinjection, chemical fusion or virus-based fusion.

In another embodiment of the present invention, the isolating step of the method of making a hybrid stem cell comprises fluorescence-activated cell sorting. In yet another embodiment of the present invention, the method of making a hybrid stem cells further comprises culturing the hybrid stem cells after the isolating step.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1 depicts adipose-derived stem cells (ADSC) isolated from TgN(GFPU)5Nagy mice in accordance with the teachings of the present invention. FIG. 1a depicts green fluorescent protein (GFP) expression in the cells by fluorescent microscopy. FIG. 1b depicts the same cells as FIG. 1a under phase contrast microscopy.

FIG. 2 depicts differentiated ADSCs made in accordance with the teachings of the present invention. Adipose-derived stem cells were induced to differentiate into five tissue types (neurogenic, adipogenic, osteogenic, chondrogenic and cardiogenic) and the differentiated and control cells assay by histology for Oil Red O (adipogenesis), Von Kossa (osteogenesis) and Alcian Blue (chondrogenesis) and by immunohistochemistry for nestin expression (neurogenesis) and cardiac tropinin I (cardiogenesis).

FIG. 3 depicts enucleation of ADSCs made in accordance with the teachings of the present invention. FIG. 3a depicts cytochalasin D-treated ADSCs post enucleation. FIG. 3b depicts control cells. FIG. 3c depicts cytochalasin D-treated ADSCs three hours post treatment.

FIG. 4 depicts stem cell hybrids two weeks post fusion made in accordance with the teachings of the present invention.

FIG. 5 depicts stem cell hybrids four weeks post fusion made in accordance with the teachings of the present invention. FIG. 5a depicts GFP positive staining cells in the stem cell hybrid cultures. FIG. 5b depicts the same cells as in FIG. 5a observed under phase contrast microscopy.

FIG. 6 depicts stem cell hybrids six weeks post fusion made in accordance with the teachings of the present invention. FIG. 6a depicts GFP positive staining cells in the stem cell hybrid cultures. FIG. 6b depicts the same cells as in FIG. 6a observed under phase contrast microscopy.

FIG. 7 depicts fluorescence-activated cell sorting (FACS) analysis of hybrid stem cells made in accordance with the teachings of the present invention. The Control (−) GFP panel depicts control cells that do not express GFP; the G3.8 hybrid panel depicts GFP expression in the G3.8 stem cell hybrid clone and the G3.9 hybrid panel depicts GFP expression in the G3.9 stem cell hybrid clone.

FIG. 8 depicts the results of single cell polymerase chain reaction (PCR) amplification of GFP from hybrid stem cell clones made in accordance with the teachings of the present invention.

FIG. 9 depicts the adipogenic differentiation of hybrid stem cells made in accordance with the teachings of the present invention.

FIG. 10 depicts the osteogenic differentiation of hybrid stem cells made in accordance with the teachings of the present invention.

FIG. 11 depicts the chondrogenic differentiation of hybrid stem cells made in accordance with the teachings of the present invention.

FIG. 12 depicts the neurogenic differentiation of hybrid stem cells made in accordance with the teachings of the present invention.

FIG. 13 depicts the cardiogenic differentiation of hybrid stem cells made in accordance with the teachings of the present invention.

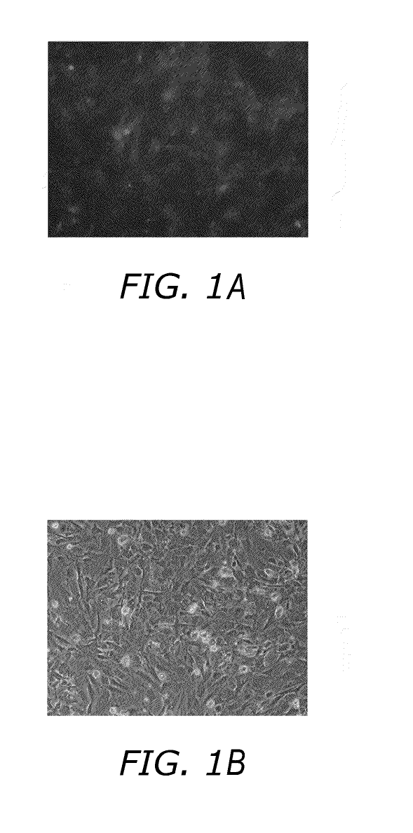

### DEFINITION OF TERMS

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Embryo: As used herein, “embryo” refers to an animal in the early stages of growth and differentiation that are characterized implantation and gastrulation, where the three germ layers are defined and established and by differentiation of the germs layers into the respective organs and organ systems. The three germ layers are the endoderm, ectoderm and mesoderm.

Embryonic Stem Cell: As used herein, “embryonic stem cell” refers to any cell that is totipotent and derived from a developing embryo that has reached the developmental stage to have attached to the uterine wall. In this context embryonic stem cell and pre-embryonic stem cell are equivalent terms. Embryonic stem cell-like (ESC-like) cells are totipotent cells not directly isolated from an embryo. ESC-like cells can be derived from primordial sex cells that have been dedifferentiated in accordance with the teachings of the present invention.

Fetal Stem Cell: As used herein, “fetal stem cell” refers to a cell that is multipotent and derived from a developing multi-cellular fetus that is no longer in early or mid-stage organogenesis.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells—blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm.

### DETAILED DESCRIPTION OF THE INVENTION

The present invention provides biologically useful pluripotent therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues.

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types (Kanatsu-Shinohara M. et al. Generation of pluripotent stem cells from neonatal mouse testis. Cell 119:1001-12, 2004).

The ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G₀/G₁ phase of the cell cycle. In mice and rats these gonocytes resume division within a few days after birth to generate spermatogonial stem cells and eventually undergo differentiation and meiosis related to spermatogenesis.

Primordial sex cells are directly responsible for generating the cells required for fertilization and eventually a new round of embryogenesis to create a new organism. Primordial sex cells are not programmed to die and are of a quality comparable to that of an embryonic state.

Embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage.

For the purpose of this discussion, an embryo and a fetus are distinguished based on the developmental stage in relation to organogenesis. The pre-embryonic stage refers to a period in which the pre-embryo is undergoing the initial stages of cleavage. Early embryogenesis is marked by implantation and gastrulation, wherein the three germ layers are defined and established. Late embryogenesis is defined by the differentiation of the germ layer derivatives into formation of respective organs and organ systems. The transition of embryo to fetus is defined by the development of most major organs and organ systems, followed by rapid fetal growth.

Embryogenesis is the developmental process wherein an oocyte fertilized by a sperm begins to divide and undergoes the first round of embryogenesis where cleavage and blastulation occur. During the second round, implantation, gastrulation and early organogenesis takes place. The third round is characterized by organogenesis and the last round of embryogenesis, wherein the embryo is no longer termed an embryo, but a fetus, is when fetal growth and development occurs.

During embryogenesis the first two tissue lineages arising from the morulae post-cleavage and compaction are the trophectoderm and the primitive endoderm, which make major contributions to the placenta and the extraembryonic yolk sac. Shortly after compaction and prior to implanting the epiblast or primitive ectoderm begins to develop.

The epiblast provides the cells that give rise to the embryo proper. Blastulation is complete upon the development of the epiblast stem cell niche wherein pluripotent cells are housed and directed to perform various developmental tasks during development, at which time the embryo emerges from the zona pellucida and implants to the uterine wall.

Implantation is followed by gastrulation and early organogenesis. By the end of the first round of organogenesis, all three germ layers will have been formed; ectoderm, mesoderm and definitive endoderm and basic body plan and organ primordia are established. Following early organogenesis, embryogenesis is marked by extensive organ development at which time completion marks the transformation of the developing embryo into a developing fetus which is characterized by fetal growth and a final round of organ development. Once embryogenesis is complete, the gestation period is ended by birth, at which time the organism has all the required organs, tissues and cellular niches to function normally and survive post-natally.

The process of embryogenesis is used to describe the global process of embryo development as it occurs, but on a cellular level embryogenesis can be described and/or demonstrated by cell maturation.

Fetal stem cells have been isolated from the fetal bone marrow (hematopoietic stem cells), fetal brain (neural stem cells) and amniotic fluid (pluripotent amniotic stem cells). In addition, stem cells have been described in both adult male and fetal tissues. Fetal stem cells serve multiple roles during the process of organogenesis and fetal development, and ultimately become part of the somatic stem cell reserve.

Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress.

There is a history of cellular therapy for the treatment of a variety of diseases but the majority of the use has been in bone marrow transplantation for hematopoietic disorders, including malignancies. In bone marrow transplantation, an individual's immune system is restored with the transplanted bone marrow from another individual. This restoration has long been attributed to the action of hematopoietic stem cells in the bone marrow.

There is increasing evidence that stem cells can be differentiated into particular cell types in vitro and shown to have the potential to be multipotent by engrafting into various tissues and transit across germ layers and as such have been the subject of much research for cellular therapy. As with conventional types of transplants, immune rejection is the limiting factor for cellular therapy. The recipient individual's phenotype and the phenotype of the donor will determine if a cell or organ transplant will be tolerated or rejected by the immune system.

Therefore, the present invention provides methods and compositions for providing functional immunocompatible stem cells for cellular regenerative/reparative therapy.

In an embodiment of the present invention, therapeutically reprogrammed cells are provided. Therapeutic reprogramming refers to a maturation process wherein a stem cell is exposed to stimulatory factors according the teachings of the present invention to yield pluripotent, multipotent or tissue-specific committed cells. The process of therapeutic reprogramming can be performed with a variety of stem cells including, but not limited to, therapeutically cloned cells, hybrid stem cells, embryonic stem cells, fetal stem cells, multipotent adult progenitor cells, adipose-derived stem cells (ADSC) and primordial sex cells.

Therapeutic reprogramming takes advantage of the fact that certain stem cells are relatively easily to obtain, such as spermatogonial stem cells and adipose-derived stem cells, and epigenetically reprograms these cells by exposure to stimulatory factors. These therapeutically reprogrammed cells have changed their maturation state to either a more committed cell lineage or a less committed cell lineage. Therapeutically reprogrammed cells are therefore capable of repairing or regenerating disease, damaged, defective or genetically impaired tissues.

Therapeutic reprogramming uses stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation.

In one specific embodiment of the present invention, primordial sex cells (PSC) are therapeutically reprogrammed. Primordial sex cells, residing in the lining of the seminiferous tubules of the testes and the lining of the ovaries (the spermatogonia and oogonia, respectively) have been determined to possess diploid (2N) genomes remarkably undamaged by to the effects of aging and cell division. Thus, PSCs possess genomes in a nearly physiologically prime state. A non-limiting example of a PSC particularly useful in an embodiment of the present invention is a spermatogonial stem cell. According to the teachings herein, therapeutically reprogrammed PSC cells are prepared for the maturation process using means similar to that experienced by stem cells present in the developing embryo and fetus during embryogenesis and organogenesis.

Therapeutically reprogrammed cells made in accordance with the teachings of the present invention can be used for therapeutic purposes as is, they can be cryopreserved for future use or they can be further matured into a more committed cell lineage in the following environments: (1) in a developing embryo, (2) in a developing fetus, (3) in a developing whole organ culture, or (4) in an in vitro cellular environment that is similar to that of embryogenesis and organogenesis.

Embodiments of the present invention provide methods for further maturing or differentiating therapeutically reprogrammed cells, stem cells and primordial sex cells into more committed cell lineages in a post-natal environment to provide more committed cells for use in cellular regenerative/reparative therapy. In addition the maturation and differentiation process provides therapeutic cells that can be used to treat or replace damaged cells in pre- and post-natal organs.

The present invention also provides for a composition termed a modified germ cell (MGC) comprising a mammalian primordial sex cell, or nucleus thereof, translocated into an enucleated ovum, wherein the PSC and the ovum are derived from the same species of animal or mammal, or a different animal or mammal. The mammalian PSC can be from any animal including, but not limited to, mice, rats, humans, non-human primates, cats, dogs, horses, pigs, cattle and sheep. In one embodiment the PSC is a mammalian spermatogonium, or nucleus thereof. In another embodiment, the PSC is a mammalian oogonium, or nucleus thereof. Alternative methods of enucleation and nucleus transfer are contemplated as being within the scope of the present invention including mechanical methods as well as methods utilizing electrical stimuli. The nucleus from any diploid precursor cell from the spermatogonia or oogonia can be used.

The MGC of the present invention is totipotent, pluripotent, multipotent or bipotent. That is, the MGC is capable of forming at least one type of tissue and more particularly, the MGC is capable of forming more than one type of tissue.

Once an MGC is generated, it can be manipulated by various methods described herein to produce a function cell capable of cellular reparative/regenerative therapy. For example, the MGC can be matured in a step-wise manner to particular stages of development typical of a mature stem cell.

In the step-wise method described herein, the MGC is first expanded to about a 6-cell stage. The MGC can be expanded to more than a 6-cell stage, however, beyond the 10-cell stage, germ cells begin to differentiate into progenitor or precursor cells. The 6-cell stage MGC is then matured in a step-wise fashion using cues from cells isolated from isolated from different gestational to post-natal stages. At least one group of cells from a gestational to post-natal donor is used to facilitate the maturing of the MGC. However, more than one group of cells may be required for a MGC to reach the desired maturation state. The mature MGC is termed a primed MGC. A primed MGC has sufficient stage-specific receptors such that, upon transplantation into a host animal or tissue, in vivo or in vitro, the primed MGC behaves similar to a mature stem cell. Methods for screening MGCs to determine the constellation of receptors expressed on their surface are well known in the field.

Additionally, MGCs and pre-embryonic, embryonic, fetal or post-natal stem cells (i.e. spermatogonial stem cells) can be matured by culturing the cells in vivo in a cellular environment containing maturation and differentiation signals appropriate for the MGC or stem cell's intended use. For example, and not intended as a limitation, embryonic stem cells mature in the embryo in the developing bone marrow niche. Blood cell development, called hematopoiesis, passes through discrete stages in specific tissues in the developing embryo before converging in the bone marrow, where it continues throughout adulthood. In a developing embryo, hematopoietic stem cell precursors develop first in the yolk sac and a region called the aorta-gonad-mesonephros. During the course of embryogenesis and organogenesis, the hematopoietic stem cell precursors migrate to the liver, and later to the spleen, before finally colonizing the bone marrow prior to birth. Therefore, hematopoietic, mesenchymal stem cells and multipotent adult progenitor cells (MAPCs) can be generated from MGCs and stem cells that can be isolated from a post-natal organism. Potential sites of in vivo maturation include, but are not limited to, sites within the developing embryo or developing fetus including the blastocyst, placenta, yolk sac, para-aortic splanchnopleura, aorta-gonad mesonephros, uterine vein or fetal liver.

One embodiment of the present invention provides MGCs generated by any animal and provides methods of using the MGCs to contribute therapeutics comprising injecting the primed MGCs into the host animal. MGCs can be derived with cells from the same species or cells from different species. Additionally primed MGCs can be transplanted into hosts of the same or different species as the component cells. The primed MGCs can be used to repair tissues to treat disease.

In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals.

In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G₀ phase) and therefore can have a methylation status than conveys greater plasticity for reprogramming.

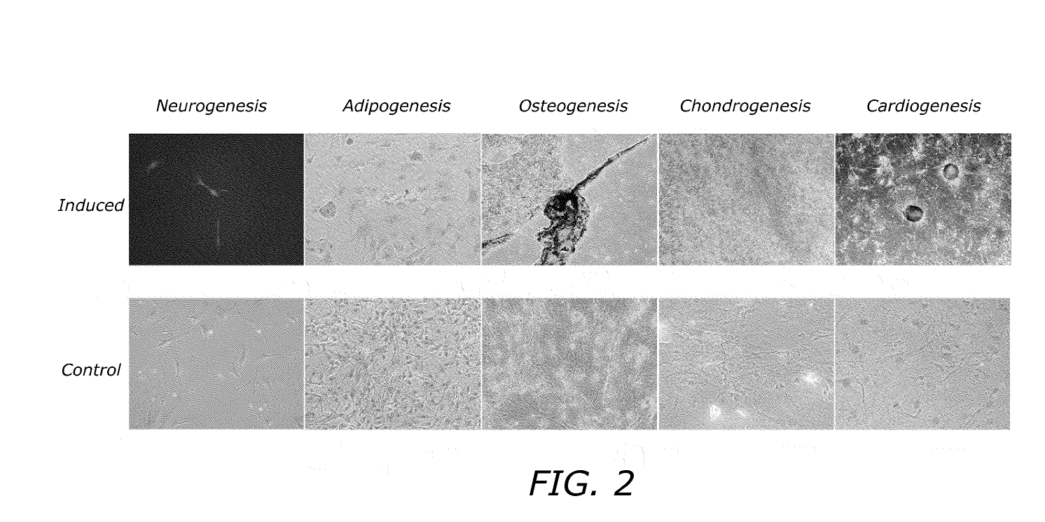

Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell types. FIG. 2 depicts the differentiation of ADSC into these five cell types. In one embodiment of the present invention, ADSCs demonstrated the greatest differentiation potential if they were differentiated prior to passage four.

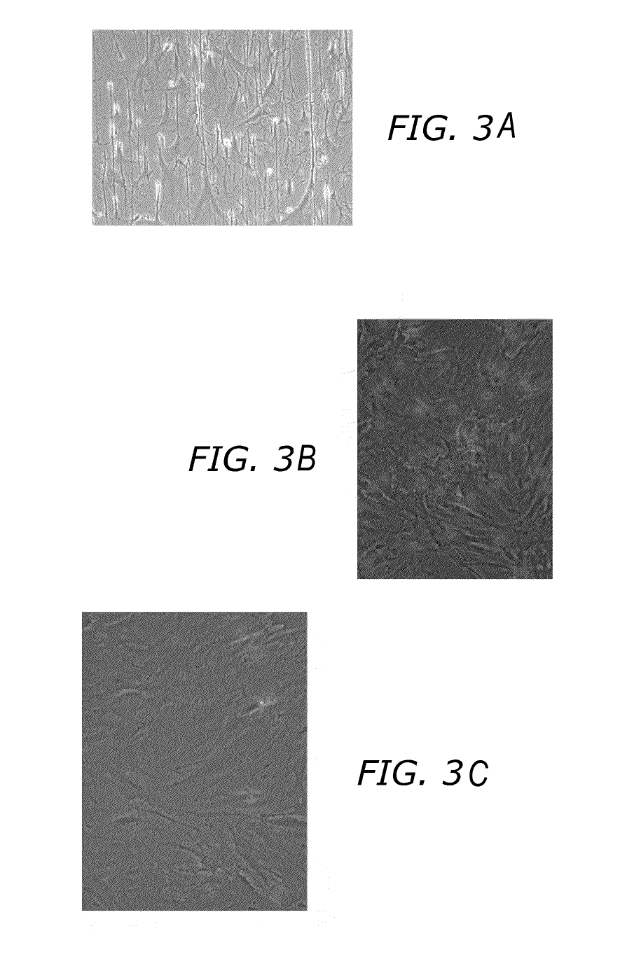

Host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. FIG. 3 depicts enucleated ADSCs made in accordance with the teachings of the present invention.

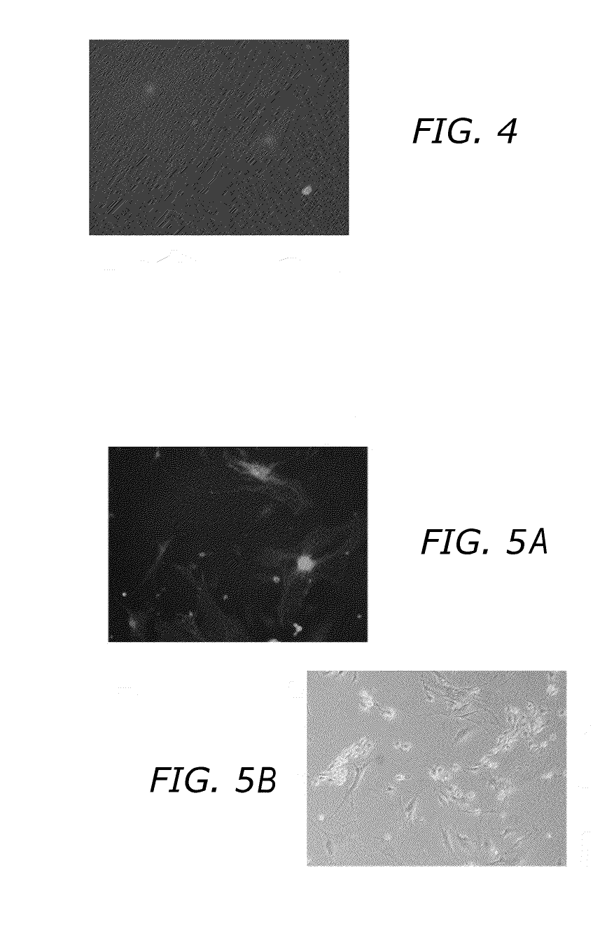

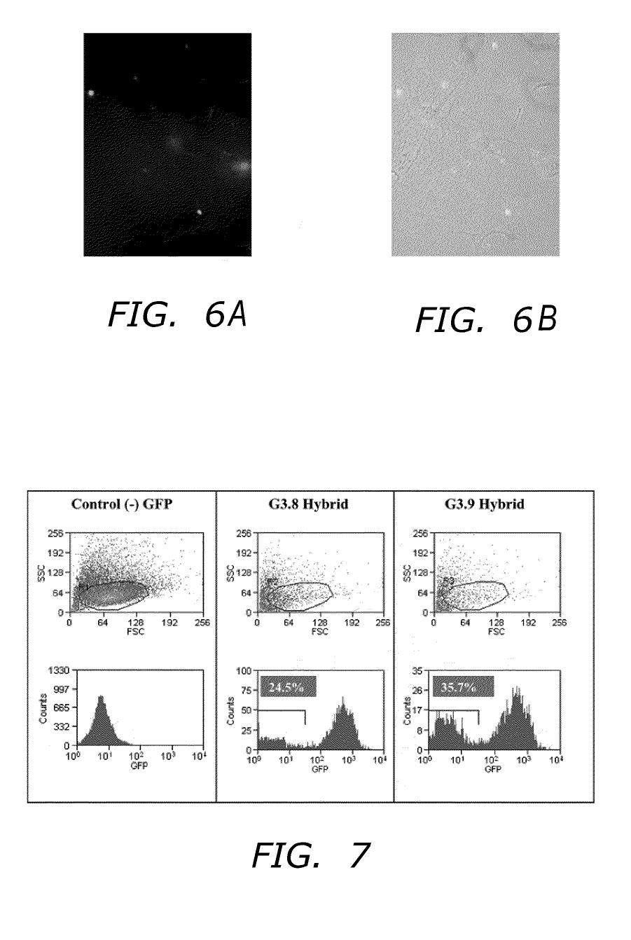

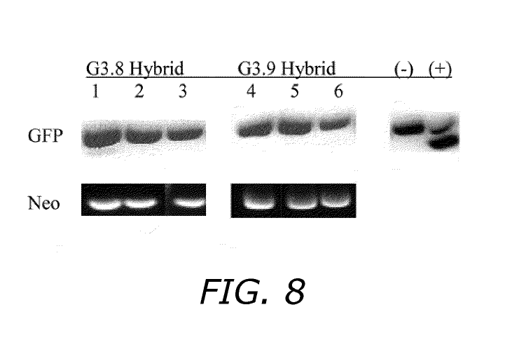

Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. FIGS. 4-6 depict hybrid stem cells made according to the teachings of the present invention from two to six weeks post-fusion demonstrating that with increased time in culture, the number of cells identified as donor cells decreases and large hybrid stem cells are seen. FIGS. 7 and 8 depict analysis of hybrid stem cells by fluorescence activated cell sorting (FACS) (FIG. 7) and polymerase-chain reaction for green fluorescent protein (GFP) expression (FIG. 8).

The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage.

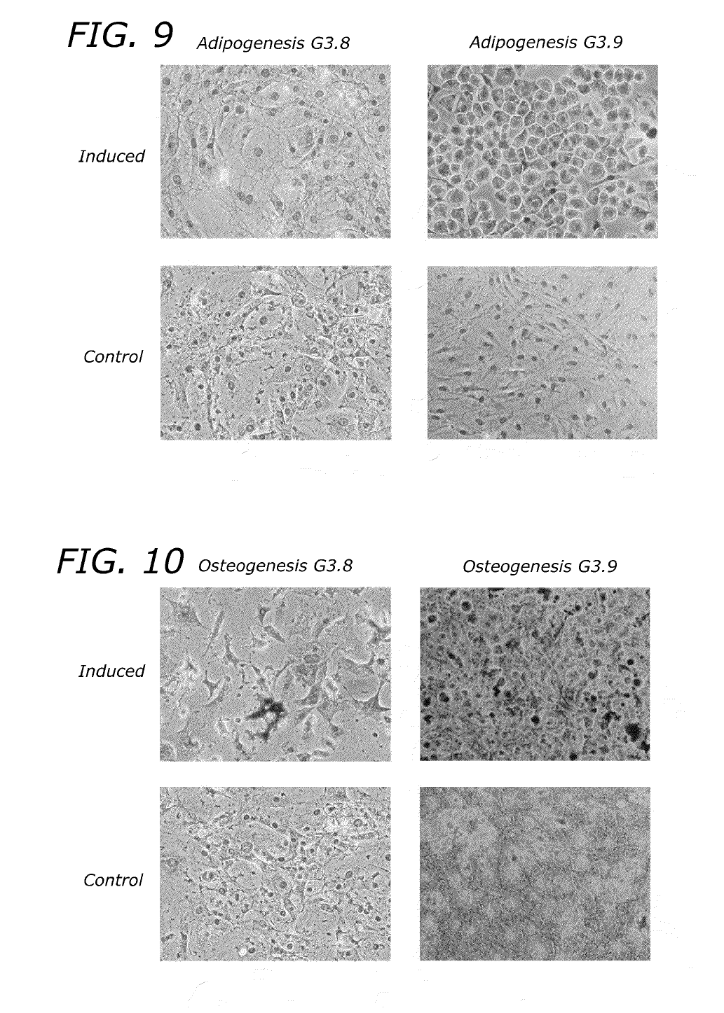

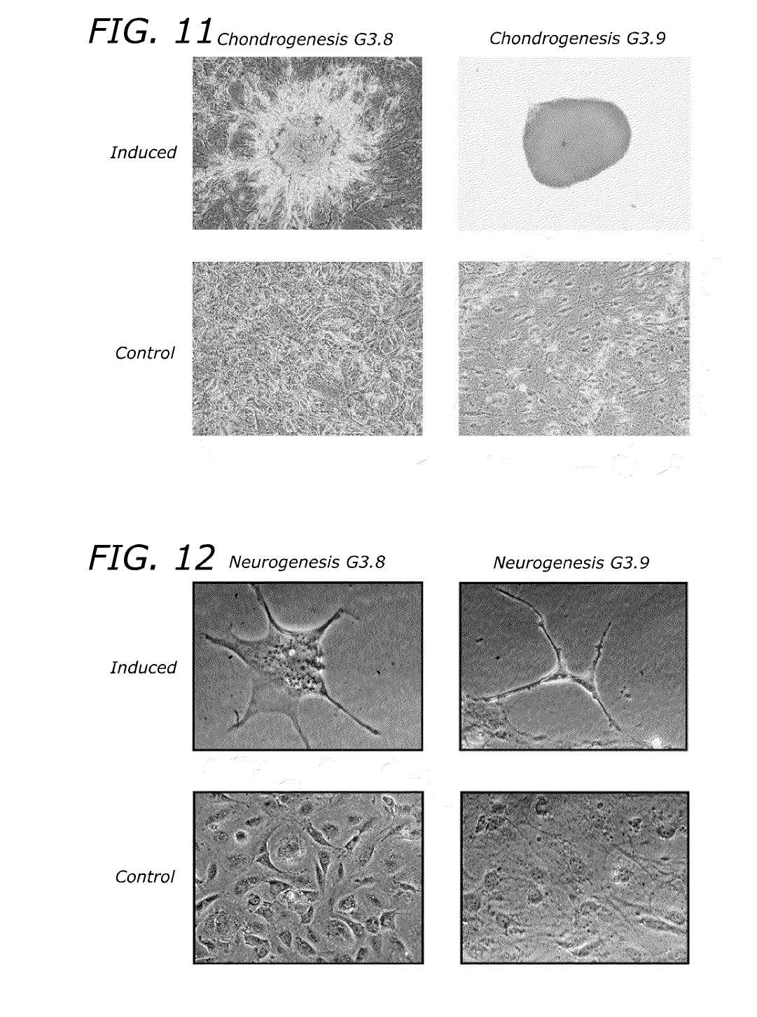

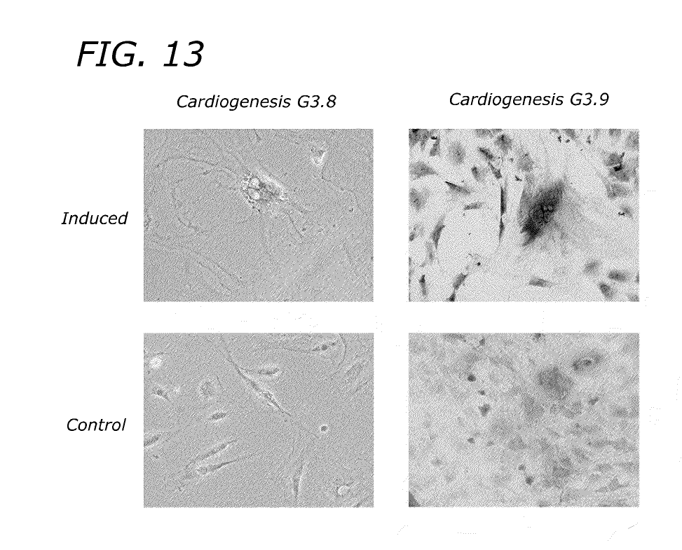

Hybrid stem cells made in accordance with the teachings of the present invention can be induced to differentiate into a variety of cell types. As an example, and not intended as a limitation to the differentiation potential of the hybrid stem cells of the present invention, hybrid stem cells can be differentiated into adipogenic cells, osteogenic cells, chondrogenic cells, neurogenic cells and cardiogenic cells. Differentiation can be performed using commercially available kits or according to methods known to persons having skill in the art. Non-limiting examples of differentiated cells generated from hybrid stem cells made according to the teachings of the present invention are depicted in FIG. 9 (adipogenic differentiation, FIG. 10 (osteogenic differentiation), FIG. 11 (chondrogenic differentiation), FIG. 12 (neurogenic differentiation) and FIG. 13 (cardiogenic differentiation).

The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

Stem cells are not universally susceptible to the maturation process of the present invention. Therefore the present inventors have developed a therapeutic reprogramming process whereby stem cells are induced into a state whereby they are susceptible to maturation factors. This therapeutic reprogramming process can be accomplished by incubation with stimulatory factors under suitable conditions and for a time sufficient to render the donor cell susceptible for maturation.

The MGCs and hybrid stem cells generated according to the methods of the present invention are also suitable for therapeutic reprogramming and maturation using the processes of the present invention. The resultant matured or differentiated MGCs, hybrid stem cells and therapeutically reprogrammed cells provide functional immunocompatible stem cells for cellular regenerative/reparative therapy.

In instances where embryonic stem cells (ESC) are used for maturation, a cell might require a preparation step in order to allow the ESC to be responsive to maturation. A non-limiting example of a preparation step in an ESC is its induction into an embryoid body or hematopoietic stem cell-like state prior to exposure to the maturation process. An embryoid body is a spheroid aggregate of embryonic stem cells that can undergo differentiation. This preparation step can also be induced by the use of chemicals or cellular extracts that influence the genomic state of the donor cell to be functional in a particular developmental period.

The following examples are meant to illustrate one or more embodiments of the invention and are not meant to limit the invention to that which is described below.

### Example 1

### Maturation

### Pre-Embryo, Embryo Transplantation

Embryonic stem cells (ESC) derived from a strain 129/SvJ mouse are injected into 3.5 days-post-conception C57BL/6J blastocysts. Within the blastocyst is the inner cell mass niche that contains the epiblast, which is responsible for germ layer establishment and ultimately all cells in the embryo. The ESC cells recognize this niche and respond by being directed appropriately to contribute to the embryo proper. After a short culture period, the blastocysts are transferred back into a pseudopregnant female and allowed to develop to term. The ESC cells under the direction of the inner cell mass and the cellular environment mature into different stem cells and support cells that are required during particular periods of embryogenesis and organogenesis. Depending on the ability of the ESC cells to respond to the maturation factors present during embryogenesis and organogenesis, chimeric mice will be born with differing levels of chimerism. Some of the mice will have a very high ESC cell contribution and some will have low levels. The ESC cells integrate to varying degrees in the respective organs and the niches that supply the cells required for organ maintenance and repair. If the ESC cells populate the germ-line niche, where the cells required for gonad maintenance and repair are located, then the resulting ESC-derived spermatogonial stem cells are able to generate gametes. When the resulting mouse chimeras are mated there are three possible outcomes: 100% germ-line contribution, where all F1 are 129/SvJ origin; a mixed germ-line contribution, where the F1 are both 129/SvJ and C57BL/6J origin; and 0% germ-line contribution where all the F1 are C57BL/6J origin. There is a niche in the gonad that is responsible for supplying the cells that contribute to the maintenance, repair and production of gametes and the presence of a mixed population in the F1 suggests that these niches allows for the possibility of two distinct populations of stem cells (129/SvJ and C57BL/6J) to co-exist. Similar to the way ESC cells populate the germ-line niche, it is also possible for the ESC cells to populate other stem cell niches such as the bone marrow, allowing the isolation of stem cells such as hematopoietic, mesenchymal or multipotent adult progenitor cells and to use them therapeutically.

### Example 2

### Maturation of Embryonic Stem Cells in the Developing Embryo

In this example, embryonic stem cells are matured in the developing bone marrow niche. Blood cell development, called hematopoiesis, passes through discrete stages in specific tissues in the developing embryo before converging in the bone marrow, where it continues throughout adulthood. In a developing embryo, hematopoietic stem cell precursors develop first in the yolk sac and a region called the aorta-gonad-mesonephros (AGM). During the course of embryogenesis and organogenesis, the hematopoietic stem cell precursers migrate to the liver, and later to the spleen, before finally colonizing the bone marrow prior to birth. In this particular example, hematopoietic, mesenchymal stem cells and multipotent adult progenitor cells (MAPCs) are generated that can be isolated from a post-natal organism.

An embryonic stem cell (ESC) is derived from a strain 129/SvJ mouse are transfected with a fluorescent reporter gene (i.e. GFP). A host C57BL/6J female mouse is mated and the day of vaginal plug discovery is designated E0.5. At a designated point in the timed pregnancy (E.7.5-E18.0), the mice are anesthetized with intraperitoneal ketamine (1.5 mg/kg) and xylazine (15 mg/kg) in 0.9% NaCl. Terbutaline (0.5 mg/kg) in 0.9% NaCl is administered subcutaneously to diminish uterine contractility. A limited low midline laparotomy is then performed and both uterine horns are externalized.

Heat-pulled glass micropipettes (Sutter Instrument Co.) with tip diameters of approximately <10-50 μm are connected to a pneumatic microinfusion pump and used to deliver approximately 1×10⁴ to approximately 1×10⁶ ESCs to a site in the embryo at 5 psi. The sites for injection of ESCs for maturation include, but are not limited to, the placenta, yolk sac, para-aortic splanchnopleura, aorta-gonad-mesonephros, uterine vein or fetal liver. The uterus is then returned to the abdomen, which is closed and the female mouse is allowed to recover and the pregnancy to go to term. At approximately 3 months post birth, the host mouse containing the transplanted ESC cells is euthanized and the femurs and tibias removed and placed in HBSS+ (Gibco-BRL)/2% FBS (Hyclone)/10 mM HEPES buffer (Gibco-BRL), on ice. The bones are cleaned free of muscle and fatty tissue and placed on ice until processing is complete. The tibias and femurs are then flushed with HBSS+/2% FBS/10 mM HEPES buffer to yield a suspension of bone marrow cells. Bone marrow mononuclear cells (BMMNC) are then collected by Ficoll-Hypaque separation. The BMMNC are plated at 1×10⁵/cm² on fibronectin- (FN; Sigma) coated dishes in MAPC media (60% DMEM-LG (Gibco BRL), 40% MCDB-201 (Sigma), 1× insulin-transferrin-selenium, 1× linoleic-acid-bovine-serum-albumin, 10⁻⁹ M dexamethasone (Sigma), 10⁻⁴M ascorbic acid 2-phosphate (Sigma), 100 units of penicillin, 1000 units of streptomycin (Gibco BRL), 2% fetal calf serum (FCS; Hyclone Laboratories), 10 ng/mL hPDGF-BB (human platelet derived growth factor-BB, R&D Systems), 10 ng/mL mEGF (mouse epidermal growth factor, Sigma) and 1000 units/mL mLIF (mouse leukemia inhibitory factor, Chemicon)). The BMMNC cultures are maintained at 5×10³/cm² and after 3-4 weeks cells are harvested and depleted of CD45⁺/Terr119⁺ cells using a micromagnetic bead separator (Miltenyi Biotec). The CD45⁻/Terr119⁻ fraction (˜20%) is plated at 10 cells per well of a FN-treated (10 ng/mL) 96-well plate and expanded at densities of 0.5-1.5×10³/cm². Approximately 1% of the wells yield continuous growing MAPC cultures. MAPCs are characterized as staining negative for CD3, Gr-1, Mac-1, CD19, CD34, CD44, CD45, cKit and major histocompatibility complex (MHC) class-I and class-II.

### Example 3

### Therapeutic Cloning and Maturation

The preparation of human primordial sex cells (donor cells) responsive to maturation signals for therapeutic cloning are described. In some instances the donor cells need an additional step to prepare for maturation. The process involved in preparing primordial sex cells (PSC) from other mammals, including humans, is similar to that described here with the possible exception of modifications to media or chemicals that are specific to that particular species.

Oocytes are collected after ovarian stimulation and matured (metaphase II) in vitro in G1.2 medium (Vitro Life, Goteborg, Sweden). Oocytes with a first polar body are selected for enucleation. Enucleation is performed in HEPES-buffered Ca²⁺-free CR2 medium with amino acids (hCR2aa) supplemented with 10% FBS and 5 ug/mL cytochalasin B (Sigma). The oocyte is held in place with a holding pipette and small slit is made on the zona pellucida with a fine needle. The first polar body and cytoplasm containing the metaphase II chromosomes are removed with a needle. Enucleation is confirmed by staining the enucleated oocytes with Hoechst 33342 (Sigma) for 5 min and observed under epifluorescence. Enucleated oocytes are then placed in HEPES-buffered TCM-199 medium (Life Technologies) supplemented with 10% FBS. Donor cells are prepared as described in Example 9. A single donor cell is placed into the perivitelline space of an enucleated oocyte treated with 100 ug/mL phytohemagglutinin (Sigma) in hCR2aa. Fusion is performed by placing the donor PSC and enucleated ovum combination in fusion medium (0.26 M mannitol, 0.1 mM MgSO₄, 0.5 mM HEPES, and 0.05% (w/v) BSA) and fused in a BTX 453, 3.2 mm gap chamber after 3 min equilibration. The fusion is induced with two DC pulses of 1.75-1.85 kV/cm for 15 sec using a BTX Electro-cell manipulator 200. The fusion product of the donor cell nucleus and the enucleated ovum now is termed a modified germ cell. The modified germ cell is then cultured for 2 hours post fusion. Activation is performed by exposing the modified germ cell to 10 μM calcium ionophore A23187 for 5 min in G1.2 medium, followed by incubation with 2.0 mM 6-dimethylaminopurine (DMAP) and incubated for 4 hours at 37° C. in 6% CO₂, 5% O₂, 89% N₂, in G1.2 medium. The modified germ cell is then washed 10 times in G1.2 medium and cultured in G1.2 medium for 48 hours followed by culture in human modified synthetic oviductal fluid (SOF) with amino acids (hmSOFaa) for 6 days. HmSOFaa was prepared by adding 10 mg/mL human serum albumin and 1.5 mM fructose to hmSOFaa. The zona pellucida is removed from the modified germ cell by digestion with 0.1% pronase (Sigma). The inner cell mass (ICM) is isolated from the modified germ cell by immunosurgery and the ICM is incubated with 100% anti-human serum antibody (Sigma) for 20 min, followed by an additional 30 min exposure to guinea pig compliment (Life Technologies) at 37° C. in 5% CO₂. The isolated ICM from the modified germ cells are cultured on mitomycin C-inactivated primary mouse embryonic fibroblast (PMEF) feeder layers in 0.1% gelatin coated 4-well tissue culture dishes. At this stage the modified germ cells mature into modified embryonic stem cells. Modified embryonic stem cells are cultured in DMEM/DMEM F12 (1:1) (Life Technologies), 0.1 mM β-mercaptoethanol (Sigma Aldrich, Corp.), 1% nonessential amino acids, 100 units/mL penicillin, 100 ug/mL streptomycin, and 4 ng/mL basic fibroblast growth factor (bFGF; Life Technologies). Additionally, up until the first passage, 2,000 units/mL of human LIF (leukemia inhibitory factor, Chemicon) is added to the medium. Karyotyping is then performed on the cells and only cell lines that are euploid are kept for maturation.

### Example 4

### Isolation of Primordial Sex Cells from Testes

The testes are excised and decapsulated. Testicular tissue is minced using fine scissors and transferred into culture medium (DMEM/F12) containing 1 mg/mL collagenase type I (Sigma) and 0.5 mg/mL DNase (Sigma). Digestion is performed at 37° C. for 10 min in a shaking water bath operated at 110 cycles/min. Interstitial cells are separated by sedimentation at unit gravity for 10 min and washed in DMEM/F12.

A final digestion of the basal lamina components of the testicular tissue is carried out in a mixture of collagenase type I (1 mg/mL), DNase (0.5 mg/mL), and hyaluronidase (Sigma; 0.5 mg/mL) under the same conditions as for the first digestion step. The single-cell suspension obtained is washed successively with medium and PBS containing 1 mM EDTA (Sigma) and 0.5% fetal calf serum. The undigested remains of the tunica albuginea are eliminated by filtering the cell suspension through a 50 μm nylon mesh. All cells are kept at 5° C. throughout the procedure. The dissociated testicular cells are suspended (5×10⁶ cells/mL) in PBS containing 0.5% FBS (PBS/FBS). The cells are then incubated with primary antibodies for 20 min on ice, washed twice with excess PBS/FBS, and used for FACS analysis. Primary antibodies include R-phycoerythrin (PE)-conjugated anti-α6-integrin, allophycocyanin (APC)-conjugated anti-c-kit, and biotinylated anti-αv-integrin. For experiments using secondary reagents, cells are further incubated for 20 min with APC-conjugated streptavidin to detect biotinylated antibody. All antibodies or secondary reagents are used at 5 μg/ml. Control cells are not treated with antibodies. After the final wash, the cells are resuspended (10⁷ cells/mL) in 2 mL PBS/FBS containing 1 μg/mL propidium iodide (Sigma), filtered into a tube through a 35 μm pore-size nylon screen, and kept in the dark on ice until analysis. The cells are sorted based on antibody staining and their relative granularity or internal complexity (side scatter, SSC). Cell sorting is performed by a dual-laser FACStar Plus (Becton Dickinson) equipped with 488-nm argon (200 mW) and 633-nm helium neon (35 mW) laser. An argon laser is used to excite PE and propidium iodide, and emissions are collected with a 575 DF 26 filter for PE and a 610 DF 20 filter for propidium iodide. A neon laser is used to excite APC, and emission is detected with a 675 DF 20 filter. Dead cells are excluded by eliminating propidium iodide-positive events at the time of data collection. Cells are sorted into 5 mL polystyrene tubes containing 2 mL of ice-cold DMEM supplemented with 10% FBS (DMEM/FBS). The α6-integrin^{hi}/SSC^{lo}/c-kit(−) population is used as the donor cell.

### Example 5

### Isolation of Primordial Sex Cells from Ovaries

The animal is anesthetized and the ovaries are removed. Alternatively, primordial sex cells (PSCs) can be isolated from a punch biopsy the ovaries. The PSCs are then isolated with the assistance of a microscope. Primordial sex cells have stem cell morphology (i.e. large, round and smooth) and are mechanically retrieved from the ovaries.

### Example 6

### Therapeutic Reprogramming with Chemical Factors

This example describes the therapeutic reprogramming of a PSC so that it is functional and responds appropriately during maturation by inducing genomic methylation changes with chemicals.

Primordial sex cells are isolated as described in Example 4. The α6-integrin^{hi}/SSC^{lo}/c-kit(−) population is used as the donor cell. The cell, or nuclear material contained therein, is then exposed to varying concentrations of DNA demethylation agents including, but not limited to, 5-aza-2′-deoxycytidine, histone deacetylase inhibitor, n-butyric acid or trichostatin A. Following genomic modification the primordial sex cell is ready to undergo a maturation process.

### Example 7

### Therapeutic Reprogramming with Whole Cell Extract Factors

This example describes the therapeutic reprogramming of a PSC so that it is functional and responds appropriately during maturation by inducing genomic methylation changes with whole cell (karyoplast/cytoplast) extracts from embryonic stem cells.

Primordial sex cells are isolated as described in Example 4. The α6-integrin^{hi}/SSC^{lo}/c-kit(−) population is used as the reprogrammable cell. These cells are stored on ice until exposure to whole cell extracts.

For preparation of whole cell extracts from embryonic stem cells (ESC), the cells are washed three times with ice-cold PBS, followed by a wash in cell lysis buffer (50 mM NaCl, 5 mM MgCl₂, 20 mM Hepes, pH 8.2, and 1 mM dithiothreitol). The cells are then centrifuged at 350×g and resuspended in 1.5 volumes of cell lysis buffer containing protease inhibitors and incubated on ice for 45 min. The cells are then homogenized by pulse sonication and the whole-cell lysates centrifuged at 16,000×g for 20 min at 4° C. The supernatant is then collected and protein concentration determined to be approximately 6 mg/mL.

The previously isolated PSCs are washed three times with ice-cold PBS, followed by a two washes in HBSS. The cells are then centrifuged at 350×g for 5 min at 4° C. and resuspended at 10,000 cells per 14 μL of ice-cold HBSS. The cells are then incubated at 37° C. for 2 min followed by the addition of streptolysin O (SLO; Sigma) at a final concentration of 115 ng/mL to 230 ng/mL depending on cell number and incubated for 50 min at 37° C. with constant shaking to keep the cells from sedimenting. The cells are then centrifuged at 500×g for 5 min at 4° C. and the supernatant removed. The PSCs are then incubated with 50 μL of previously prepared embryonic stem cell whole cell extract containing an ATP-regenerating system and 1 mM of each of the four nucleoside triphosphates (NTP) at 37° C. for 1-2 hours. The cells are then resuspended in solution of 2 mM CaCl₂ in preparation media (1% nonessential amino acids, 1% L-glutamine, 100 units/mL penicillin, 100 μg/mL streptomycin, 0.1 mM β-mercaptoethanol, 3,000 units/mL of LIF in DMEM/20% FBS) and placed into one well of a 48-well dish pre-treated with 0.1% gelatin containing a mitomycin C-inactivated primary embryonic fibroblast (PEF) layer. In addition, it is also possible to co-culture the extract-treated PSCs in a 48-well dish pre-treated with 0.1% gelatin containing a mitomycin C-inactivated PEF layer and 50% confluent ESCs. After 24 hours, cells that were not attached to the feeder layer were removed and the extract exposure procedure was repeated a second time with the unattached cells. The reprogrammed cells (attached cells) are cultured and assayed for embryonic stem cell specific markers (i.e. REX1, OCT4), and tested for in vitro differentiation potential prior to being exposed to a maturation process.

### Example 8

### Therapeutic Reprogramming with Cytoplast Extract Factors

This example describes the therapeutic reprogramming of a PSC so that is functional and responds appropriately during maturation by inducing genomic modifications using cytoplast extracts from embryonic stem cells.

Primordial sex cells were isolated as described in Example 4. The α6-integrin^{hi}/SSC^{lo}/c-kit(−) population is used as the reprogrammable cell. These cells are stored on ice until exposure to cytoplast extracts.

For preparation of embryonic stem cell extracts, the ESCs are cultured to confluency. The ESC cytoplasts are prepared using a discontinuous density gradient of Ficoll-400 (30%, 25%, 22%, 18% and 15%) containing 10 μg/mL cytochalasin B. Ten million ESCs in 12.5% Ficoll-400 are carefully layered on top of the gradient and centrifuged at 40,000 rpm at 36° C. for 30 min. The cytoplasts are collected from the 15% and/or the 18% levels. The cytoplasts are then washed three times with ice-cold PBS followed by a wash in cell lysis buffer. The cytoplasts are then centrifuged at 350×g and resuspended in 1.5 volumes of cell lysis buffer containing protease inhibitors and incubated on ice for 45 min. The cytoplasts are then homogenized by pulse sonication and then the cytoplasts are centrifuged at 16,000×g for 20 min at 4° C. The supernatant is then collected and protein concentration determined to be approximately 6 mg/mL.

The previously isolated PSCs are incubated with cytoplast extracts according to the methods presented in Example 7.

### Example 9

### Therapeutic Reprogramming with Karyoplast Extract Factors

This example describes the therapeutic reprogramming of a PSC so that it is functional and responds appropriately during maturation by inducing genomic modifications using nuclear (karyoplast) extracts from embryonic stem cells.

Primordial sex cells were isolated as described in Example 4. The α6-integrin^{hi}/SSC^{lo}/c-kit(−) population is used as the reprogrammable cell. These cells were stored on ice until exposure to nuclear extracts.

For preparation of embryonic stem cell nuclear (karyoplast) extracts, the ESCs are cultured to confluency. The ESC karyoplast are prepared using a discontinuous density gradient of Ficoll-400 (30%, 25%, 22%, 18% and 15%) containing 10 μg/mL cytochalasin B. Ten million ESCs in 12.5% Ficoll-400 are carefully layered on top of the gradient and centrifuged at 40,000 rpm at 36° C. for 30 min. The karyoplasts are collected from the 30% level. The karyoplasts are then washed three times with ice-cold PBS followed by a wash in cell lysis buffer. The karyoplasts are then centrifuged at 350×g and resuspended in 1.5 volumes of cell lysis buffer containing protease inhibitors and incubated on ice for 45 min. The karyoplasts are then homogenized by pulse sonication and then the karyoplasts are centrifuged at 16,000×g for 20 min at 4° C. The supernatant is then collected and protein concentration determined to be approximately 6 mg/mL.

The previously isolated PSCs are incubated with karyoplast extracts according to the methods of Example 7.

### Example 10

### Hybrid Stem Cell Creation

This example describes the generation of a hybrid stem cell. The processes presented in this embodiment can be applied to generate a hybrid stem cell using any enucleated (pre-embryonic, embryonic, fetal, or post-natal) stem cell as the host and using a PSC or any cell (pre-embryonic, embryonic, fetal, or post-natal) as the donor with the only limitation being that the donor cell be diploid (2N). In addition the donor cell, or nucleus thereof, can be genetically modified to correct a genetic dysfunction and deliver the corrected gene or transgene via stem cell-based therapy. Donor cells and host cells can be fused by methods including, but not limited to, electrical, viral, chemical or mechanical fusion. Additionally host cells can be enucleated by methods including, but not limited to, chemical, x-ray irradiation, laser irradiation or mechanical means.

Primordial stem cells are isolated as described in Example 4. The α6-integrin^{hi}/SSC^{lo}/c-kit(−) population is used as the donor cell. These cells were stored on ice until fusion with the enucleated embryonic stem cell.

For preparation of embryonic stem cell cytoplasts the ESCs are cultured until confluency. Embryonic stem cell cytoplasts are then prepared by using a discontinous density gradient of Ficoll-400 (30%, 25%, 22%, 18%, and 15%) containing 10 μg/mL cytochalasin B. Ten million ESCs in 12.5% Ficoll-400 were carefully layered on top of the gradient and centrifuged at 40,000 rpm at 36° C. for 30 minutes. The cytoplasts were collected from the 15% and/or 18% regions and stored on ice until cell fusion.

The donor cell (PSC), or nucleus thereof, is washed in cytopulse fusion medium (CytoPulse) three times and resuspended at 5×10⁶ cells, or nuclei, in 150 μL ice-cold cytopulse fusion medium. The enucleated host cells (ESCs) are washed three times in cytopulse fusion medium and resuspended at 1×10⁶ cells in 150 μL ice-cold cytopulse fusion medium. The two cell populations are mixed gently and placed in a Cytopulse fusion chamber and electrofused with following parameters: pre-sine, beginning voltage: 65 volts, duration: 50 volts, frequency: 0.8 kHz, end volts: 65 volts; pulse, amplitude: 200 volts, duration: 0.05 milli-seconds; and post-sine, beginning voltage: 65 volts, duration: 50 seconds, frequency: 0.8 kHz, end voltage: 5 volts. The cells are then allowed to recover for 30 min at 37° C. while remaining in the chamber. At 15 min post fusion, FBS is added to a final serum concentration of 10% and incubated for an additional 15 min. The fused cells are then removed and washed one time in DPBS/20% serum by centrifugation at room temperature at 500×g for 5 min and resuspended in preparation media. The fused cells are then placed into wells of a 48-well dish pre-treated with 0.1% gelatin containing a mitomycin C-inactivated PEF layer. In addition, it is also possible to co-culture the stem cell hybrids in a 48-well dish pre-treated with 0.1% gelatin containing a mitomycin C-inactivated PEF layer and 50% confluent ESCs. The fused cells are expanded for several passages to determine hybrid stem cell stability and donor cell genomic reprogramming. The hybrid stem cells are then karyotyped and only cell lines that are euploid are kept for maturation.

In one experiment, adipose-derived stem cells (ADSC) were enucleated from TgN(GFPU)5Nagy mice which constitutively express green fluorescent protein (GFP) and the cytoplasts were fused by electrofusion to lymphocytes from R26R mice. This strain of mice was chosen as the source of lymphocytes for this experiment solely due to the presence of the Neo marker in their nuclei. The presence of GFP in the host cell allows the tracking of the host nucleus. Hybrid stem cells generated by this fusion were cultured and assayed for the presence of GFP (indicating the presence of a nucleated host cell and not a stem cell hybrid). Within two weeks post fusion, individual GFP(−) cells, presumable fusion products, can be seen in culture (FIG. 4) and within four weeks colonies of GFP(−) cells were present (FIG. 5). These cells were sorted for GFP(−) cells (FIG. 7) and expanded in culture.

The hybrid stem cells produced in the above described embodiment of the present invention were further characterized for fluorescence activated cell sorting (FACS) for the presence of GFP (host nucleus) and Neo (donor nucleus). Hybrid stem cells were confirmed to be hybrids of a donor nucleus and an enucleated host cell by single cell polymerase chain reaction analysis (FIG. 8).

### Example 11

### Embryoid Body Generation

Previously isolated ESCs are induced to form embryoid bodies by withdrawing LIF from the culture medium. Aggregation is induced by placing 20 μL drops of 1,200 cells each on the lid of a non-adherent tissue culture dish which is then inverted sterile PBS. The culture medium is supplemented with fibroblast growth factor 2 and vascular endothelial growth factor A165. The day that LIF is removed from the medium and droplets formed is day 0. The droplets are left hanging on the culture dish lid for 3-5 days in an environment of 37° C. and 5% CO₂. After 3-5 days the droplets are each transferred to a well of an 8-well glass culture slide. All analyses are performed on four or more embryoid bodies at three or more individual times.

### Example 12

### Repair of Infracted Myocardium with Matured Stem Cells

The following example describes the process wherein a therapeutically reprogrammed PSC derived from a post-natal source is matured in a xenograft fetal sheep model into a post-natal stem cell and used in cell-based therapy to repair infracted myocardium. In addition to the use of freshly-isolated PSCs, frozen or banked stem cells can also be used.

Primordial sex cells are isolated as described in Example 4. The α6-integrin^{hi}/SSC^{lo}/c-kit(−) population is used as the donor cell. The donor cell, or nuclear material therein, is therapeutically reprogrammed by exposure to varying concentrations of DNA demethylation agents such as 5-aza-2′-deoxycytidine, histone deacetylase inhibitor, n-butyric acid or trichostatin A. Following demethylation, the therapeutically reprogrammed PSC is ready to undergo a maturation process, in this example therapeutic cloning.

Oocytes are collected after ovarian stimulation and matured (metaphase II) in vitro in G1.2 medium. Oocytes with a first polar body are selected for enucleation. Enucleation is performed in hCR2aa supplemented with 10% FBS and 5 ug/mL cytochalasin B. The oocyte is held in place with a holding pipette and small slit is made on the zona pellucida with a fine needle. The first polar body and cytoplasm containing the metaphase II chromosomes are removed with a needle. Enucleation is confirmed by staining the enucleated oocytes with Hoechst 33342 for 5 min and observed under epifluorescence. Enucleated oocytes are then placed in HEPES-buffered TCM-199 medium supplemented with 10% FBS. Donor cells are prepared as previously described in Example 9. A single donor cell is placed into the perivitelline space of an enucleated oocyte treated with 100 ug/mL phytohemagglutinin in hCR2aa. Fusion is performed by placing the donor PSC and enucleated host cell combination in fusion medium (0.26 M mannitol, 0.1 mM MgSO₄, 0.5 mM HEPES, and 0.05% (w/v) BSA) and fused in a BTX 453, 3.2 mm gap chamber after 3 min equilibration. The fusion is induced with two DC pulses of 1.75-1.85 kV/cm for 15 sec using a BTX Electro-cell manipulator 200. The fusion product of the donor cell and the enucleated host cell now is termed a modified germ cell. The modified germ cell is then cultured for 2 hours post fusion. Activation is performed by exposing the modified germ cell to 10 μM calcium ionophore A23187 for 5 min in G1.2 medium, followed by incubation with 2.0 mM DMAP and incubated for 4 hours at 37° C. in 6% CO₂, 5% O₂, 89% N₂, in G1.2 medium. The modified germ cell is then washed 10 times in G1.2 medium and cultured in G1.2 medium for 48 hours followed by culture in human modified SOF with amino acids (hmSOFaa) for 6 days. HmSOFaa was prepared by adding 10 mg/mL human serum albumin and 1.5 mM fructose to hmSOFaa. The zona pellucida is removed from the modified germ cell by digestion with 0.1% pronase. The ICM is then isolated from the modified germ cell by immunosurgery and the ICM is incubated with 100% anti-human serum antibody for 20 min, followed by an additional 30 min exposure to guinea pig compliment at 37° C. in 5% CO₂. The isolated ICM from the modified germ cells are cultured on mitomycin C-inactivated PEF feeder layers in 0.1% gelatin coated 4-well tissue culture dishes. At this stage the modified germ cells mature into modified ESCs. Modified ESCs are cultured in DMEM/DMEM F12 (1:1), 0.1 mM β-mercaptoethanol, 1% nonessential amino acids, 100 units/mL penicillin, 100 ug/mL streptomycin, and 4 ng/mL bFGF. Additionally, up until the first passage, 2,000 units/mL of human LIF is added to the medium. Karyotyping is then performed on the cells and only cell lines that are euploid are kept for maturation.

In some instances the ESC might have to undergo a preparation step prior to maturation. A non-limiting example is the case of an ESC induced into an embryoid body or a hematopoietic stem cell-like condition prior to exposure to the maturation process. Additionally, the maturation preparation might be induced by means including, but not limited to, chemical, biochemical, or cellular extract (cytoplast and/or nuclear) exposure of the embryonic stem cell, or nucleus thereof.

One million male ESCs are injected into preimmune (day 48-62 of gestation) female fetal sheep recipients using the amniotic bubble procedure. Briefly, after a 48-hour fasting period, maternal ewes are injected with ketamine (10 mg/kg, intramuscularly), and receive 0.5-1.0% halothane-oxygen mixture by inhalation via an endotrachael tube. The external jugular vein is cannulated for administration of fluids and antibiotics (2 million U penicillin and 400 mg kanamycin). The uterus is exposed through a midline incision and the myometrial layers divided with electrocautery, leaving the amnion intact. The fetus is manipulated within the amniotic sac and, under direct visualization, the embryonic stem cells are injected into the fetal peritoneal cavity. The uterine and maternal body walls are closed and the fetus is allowed to go to term.

At approximately three months post birth, the host sheep containing the transplanted embryonic stem cells is euthanized. Mononuclear bone marrow cells (BMCs) are isolated by Ficoll density separation on Lymphocyte Separation Medium (BioWhittaker) before the erythrocytes are lysed with H₂O. Male cells are selected by the presence of a Y chromosome and 1×10⁶ BMCs/mL are placed in Teflon bags (Vuelife, Cell Genix) and cultivated in X-Vivo 15 medium (BioWhittaker) supplemented with 2% heat-inactivated autologous plasma. The next day, BMCs are harvested and washed three times with heparinized saline before final resuspension in heparinized saline. Viability is determined to be approximately 93±3%. The cells are heparinized and filtered to prevent cell clotting and microembolization during intracoronary transplantation. The mean number of mononuclear cells harvested after overnight culture is 2.8×10⁷, this consists of 0.65±0.4% AC133-positive cells and 2.1±0.28% CD34-positive cells. All microbiological tests of the clinically used cell preparations prove to be negative. As a viability and quality ex vivo control, 1×10⁵ cells grown in H5100 medium (Stem Cell Technology) are found to be able to generate mesenchymal cells in culture. The BMC cells are frozen and stored in a cell bank for future use.

At the time of a cardiac infarct, the cryopreserved cells are thawed and cultured. Five to nine days after onset of acute infarction, the cells are directly transplanted into the infracted zone. This is accomplished with the use of a balloon catheter placed within the infarct-related artery. After positioning of the balloon at the site of the former infarct-vessel occlusion, percutaneous transluminal coronary angioplasty (PTCA) is performed 6 to 7 times for 2 to 4 min each. During this time, intracoronary cell transplantation via the balloon catheter is performed using 6 to 7 fractional high-pressure infusions of 2 to 3 mL cell suspension, each of which contains approximately 1.5-4×10⁶ mononuclear cells. Angioplasty thoroughly prevents the backflow of cells and at the same time produced a stop-flow beyond the site of the balloon inflation to facilitates high-pressure infusion of cells into the infracted zone. Thus, prolonged contact time for cellular migration is allowed.

### Example 13

### Generation of Adipose-Derived Hybrid Stem Cells

The following is a brief description for the preparation of a hybrid stem cell so that it is functional and responds appropriately in cell-based therapies. This hybrid stem cell is derived from an enucleated adipose-derived stem cell (host cell) and a PSC, or nucleus thereof (donor cell). The adipose-derived stem cell (ADSC) can be optionally therapeutically reprogrammed before acting as a donor cell for the hybrid stem cell.

Adipose-derived stem cells were derived from a 129/SvJ mouse. Briefly, visceral fat encasing the stomach and intestines was removed and finely minced with sterile scissors. The dissected fat was then washed three times with an equal volume of calcium/magnesium-free Dulbecco's phosphate-buffered saline (DPBS-) and centrifuged at 500×g for 5 min after each wash step to remove floating adipocytes. Type I collagenase (0.075%, Sigma) was added to the minced adipose tissue and the mixture was incubated at 37° C. for 30 min with gentle agitation and an equal volume of DMEM containing 10% FBS was added to the mixture. The mixture was then centrifuged at 500×g for 10 min and the cellular pellet resuspended in DMEM containing 10% FBS. The mixture was then filtered through a 100 μm nylon mesh, centrifuged at 500×g for 10 min and resuspended in DMEM containing 10% FBS and 1× antibiotic/antimycotic (basal media). The cells were then cultured for four passages and plated onto 10 ng/mL fibronectin-coated 25×75 mm tissue culture slides. On the day of hybrid stem cell creation, 2 μg/mL of cytochalasin D (final concentration) was added to the media and the slides were incubated for 120 min at 37° C. Following the 120 min incubation step, the slides were centrifuged in a swinging bucket centrifuge at 10,000×g for one hour in basal media. After the two hour recovery period, the cells were trypsinized and prepared for cell fusion.

Primordial sex cells were prepared as described in Example 4 and the α6-integrin^{hi}/SSC^{lo}/c-kit(−) population was used as the donor cell. The donor cell, or nucleus thereof, was washed in cytopulse fusion medium (CytoPulse) three times and resuspended at 5×10⁶ cells in 150 μL ice-cold cytopulse fusion medium. The previously isolated enucleated host cells (adipose-derived stem cells) were trypsinized from the slides and washed three times in cytopulse fusion medium and resuspended at 1×10⁶ cells in 150 μL ice-cold cytopulse fusion medium. The two cell populations were mixed gently and placed in a Cytopulse fusion chamber and electrofused with following parameters: pre-sine, beginning voltage: 65 volts, duration: 50 volts, frequency: 0.8 kHz, end volts: 65 volts; pulse: amplitude: 200 volts, duration: 0.05 milli-seconds; and post-sine, beginning voltage: 65 volts, duration: 50 seconds, frequency: 0.8 kHz, end voltage: 5 volts. The cells were then allowed to recover for 30 min at 37° C. while remaining in the chamber, at 15 min post fusion FBS was added to a final serum concentration of 10% and incubated for an additional 15 min. The fused cells were then removed and washed one time in DPBS/20% serum and resuspended in basal medium.

### Example 14

### Generation of Multipotent Adult Progenitor Hybrid Stem Cells

The following is a brief description for the preparation of a hybrid stem cell that is functional and responds appropriately in cell-based therapies. This hybrid stem cell is derived from an enucleated multipotent adult progenitor cell (the host) and a PSC, or nucleus thereof (the donor cell). The multipotent adult progenitor cell (MAPC) can be optionally therapeutically reprogrammed before acting as a donor cell for the hybrid stem cell.

Bone marrow cells (BMC) are collected and resuspended in culture media and kept on ice. Bone marrow mononuclear cells (BMMNC) are isolated by Ficoll-Hypaque separation and plated at 1×10⁵/cm² on fibronectin-coated dishes in MAPC media. The BMMNC cultures are maintained at 5×10³/cm² and after 3-4 weeks cells are harvested and depleted of CD45⁺/Terr119⁺ cells using a micromagnetic bead separator. The CD45⁻/Terr119⁻ population (˜20%) is plated at 10 cells per well of a FN-treated 96-well dish and expanded at densities of 0.5-1.5×10³/cm². Approximately 1% of the wells will yield continuous growing MAPC cultures. These cells are then expanded for enucleation by plating onto fibronectin-coated 25×75 mm tissue culture slides. On the day of hybrid stem cell creation, 2 μg/mL of cytochalasin D (final concentration) is added to the media and the slides are incubated for 120 min at 37° C. Following the 120 min incubation step, the slides are centrifuged in a swinging bucket centrifuge at 10,000×g for one hour in MAPC media. After the two hour recovery period the cells are trypsinized and prepared for cell fusion.

The donor cells (PSC) are prepared as described in Example 4 and the α6-integrin^{hi}/SSC^{lo}/c-kit(−) population is used as the donor cell. The donor cell, or nucleus thereof, is washed in cytopulse fusion medium three times and resuspended at 5×10⁶ cells in 150 μL ice-cold cytopulse fusion medium. The previously isolated enucleated host cells (MAPCs) are trypsinized from the slides and washed three times in cytopulse fusion medium and resuspended at 1×10⁶ cells in 150 μL ice-cold cytopulse fusion medium. The two cell populations are mixed gently and placed in a Cytopulse fusion chamber and electrofused with following parameters: pre-sine, beginning voltage: 65 volts, duration: 50 volts, frequency: 0.8 kHz, end volts: 65 volts; pulse: amplitude: 200 volts, duration: 0.05 milliseconds; and post-sine, beginning voltage: 65 volts, duration: 50 seconds, frequency: 0.8 kHz, end voltage: 5 volts. The cells are then allowed to recover for 30 min at 37° C. while remaining in the chamber, at 15 min post fusion FBS is added to a final serum concentration of 10% and the cells are incubated for an additional 15 min. The fused cells are then removed and washed one time in DPBS/20% serum and resuspended in MAPC medium.

### Example 15

### Repair of Infracted Myocardium with Hybrid Stem Cells

The following describes the process wherein a hybrid stem cell is used in cell-based therapy to repair infracted myocardium. In this example the patient is at high-risk for a cardiac infarct. The hybrid stem cell is derived from an enucleated host cell (bone marrow cell) and a post-natal donor cell (PSC). The host cell can be obtained from a patient or from a stem cell bank or any other source, there is no concern of HLA type immune rejection since the hybrid stem cell created will contain the genomic material from the PSC of the patient.

Isolation of post-natal donor cells is as described in Example 4 and the α6-integrin^{hi}/SSC^{lo}/c-kit(−) population is used as the donor cell. In some instances the donor cell, or nucleus thereof, can undergo a preparation step prior to fusion with the host cell, to make it more receptive to the host cytoplasm. This preparation step can also include, but is not limited to, induction by chemicals, biochemicals or cellular extracts that influence the genomic state of the donor cell to be functional and receptive to the host cytoplasm.

Mononuclear bone marrow cells (BMCs) are isolated by Ficoll density separation on Lymphocyte Separation Medium before the erythrocytes are lysed with H₂O. For overnight cultivation, 1×10⁶ BMCs/mL are placed in Teflon bags and cultivated in X-Vivo 15 medium supplemented with 2% heat-inactivated autologous plasma. The next day, BMCs are harvested and washed three times with heparinized saline before final resuspension in heparinized saline. Viability is about 93±3%. Heparinization and filtration are carried out to prevent cell clotting and microembolization during intracoronary transplantation. The mean number of mononuclear cells harvested after overnight culture is approximately 2.8×10⁷; this consists of 0.65±0.4% AC133-positive cells and 2.1±0.28% CD34-positive cells. Microbiological tests of the cell preparations are negative. As a viability and quality ex vivo control, 1×10⁵ cells grown in H5100 medium are found to be able to generate mesenchymal cells in culture.

Fresh or previously cryopreserved host cells are then cultured and plated onto fibronectin coated 25×75 mm tissue culture slides. On the day of hybrid stem cell creation, 2 μg/mL of cytochalasin D (final concentration) is added to the media and the slides are incubated for 120 min at 37° C. Following the 120 min incubation step, the slides are centrifuged in a swinging bucket centrifuge at 10,000×g for 1 hour in X-Vivo 15 medium supplemented with 2% heat-inactivated autologous plasma or H5100 medium containing 2 ug of cytochalasin D. After a two hour recovery period, the cells are trypsinized and prepared for cell fusion. The host cells are trypsinized from the slides and prepared for fusion. The donor cell, or nucleus thereof, is washed in cytopulse fusion medium three times and resuspended at 5×10⁶ cells in 150 μL ice-cold cytopulse fusion medium. The enucleated host cells (BMCs) are washed three times in cytopulse fusion medium and resuspended at 1×10⁶ cells in 150 μL ice-cold cytopulse fusion medium. The two cell populations are mixed gently and placed in a Cytopulse fusion chamber and electrofused with following parameters: pre-sine, beginning voltage: 65 volts, duration: 50 volts, frequency: 0.8 kHz, end volts: 65 volts; pulse, amplitude: 200 volts, duration: 0.05 milli-seconds; and post-sine, beginning voltage: 65 volts, duration: 50 seconds, frequency: 0.8 kHz, end voltage: 5 volts. The cells are then allowed to recover for 30 min at 37° C. while remaining in the chamber, at 15 min post fusion FBS is added to a final serum concentration of 10% and the cells are incubated for an additional 15 min. The fused cells are then removed and washed one time in DPBS/20% serum and resuspended in X-Vivo 15 medium supplemented with 2% heat-inactivated autologous plasma or H5100 medium. The cells are cultured and expanded to test for HLA-type compatibility. The cells are then frozen and stored in cell bank for future cell therapy use.

At the time of cardiac infarct the cryopreserved hybrids stem cells are thawed and cultured. Five to nine days after onset of acute infarction, the cells are directly transplanted into the infracted zone. This is accomplished with the use of a balloon catheter placed within the infarct-related artery. After exact positioning of the balloon at the site of the former infarct-vessel occlusion, percutaneous transluminal coronary angioplasty (PTCA) is performed 6 to 7 times for 2 to 4 minutes each. During this time, intracoronary cell transplantation via the balloon catheter is performed, using 6 to 7 fractional high-pressure infusions of 2 to 3 mL of cell suspension, each of which contains approximately 1.5-4×10⁶ cells. Angioplasty thoroughly prevents the backflow of cells and at the same time produces a stop-flow beyond the site of the balloon inflation to facilitate high-pressure infusion of cells into the infracted zone. Thus, prolonged contact time for cellular migration is allowed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A therapeutic reprogramming method comprising:
isolating a stem cell;
contacting said stem cell with a medium comprising stimulatory factors which induce development of said stem cell into a therapeutically reprogrammed cell;
recovering said therapeutically reprogrammed cell from said medium; and
implanting said therapeutically reprogrammed cell, or a cell matured therefrom, into a host in need of a therapeutically reprogrammed cell.

2. The therapeutic reprogramming method of claim 1 wherein said stem cell is selected from the group consisting of embryonic stem cells, fetal stem cells, somatic stem cells, multipotent adult progenitor cells, modified germ cells, adipose-derived stem cells and primordial sex cells.

3. The therapeutic reprogramming method of claim 2 wherein said stem cell is an embryonic stem cell.

4. The therapeutic reprogramming method of claim 2 wherein said stem cell is a fetal stem cell.

5. The therapeutic reprogramming method of claim 2 wherein said stem cell is a somatic stem cell.

6. The therapeutic reprogramming method of claim 2 wherein said stem cell is a multipotent adult progenitor cell.

7. The therapeutic reprogramming method of claim 2 wherein said stem cell is a modified germ cell.

8. The therapeutic reprogramming method of claim 2 wherein said stem cell is an adipose-derived stem cell.

9. The therapeutic reprogramming method of claim 2 wherein said stem cell is a primordial sex cell.

10. The therapeutic reprogramming method of claim 9 wherein said primordial sex cell is a spermatogonial stem cell.

11. The therapeutic reprogramming method of claim 1 wherein said stimulatory factor is selected from the group consisting of chemicals, biochemicals, and cellular extracts.

12. The therapeutic reprogramming method of claim 11 wherein said stimulatory factor is a chemical selected from the group consisting of 5-aza-2′-deoxycytidine, histone deacetylase inhibitor, n-butyric acid and trichostatin A.

13. The therapeutic reprogramming method of claim 12 wherein said chemical is 5-aza-2′-deoxycytidine.

14. The therapeutic reprogramming method of claim 11 wherein said stimulatory factor is a cellular extract selected from the group consisting of whole cell extracts, cytoplast extracts and karyoplast extracts.

15. The therapeutic reprogramming method of claim 14 wherein said stimulatory factor is a karyoplast extract.

16. The therapeutic reprogramming method of claim 14 wherein said cellular extract is isolated from a stem cell.

17. The therapeutic reprogramming method of claim 16 wherein said stem cell is selected from the group consisting of embryonic stem cells, fetal neural stem cells, multipotent adult progenitor cells, and primordial sex cells.

18. The therapeutic reprogramming method of claim 1 wherein said host is a mammal.

19. The therapeutic reprogramming method of claim 18 wherein said mammal is a human.

20. The therapeutic reprogramming method of claim 1 wherein said stem cell is isolated from said host.

21. The therapeutic reprogramming method of claim 1 further comprising the step of maturing said therapeutically reprogrammed cell to become committed to a tissue-specific lineage.

22. A therapeutically reprogrammed cell comprising:
an SSC which has been exposed to stimulatory factors which have caused said SSC to mature or differentiate into a totipotent or a pluripotent cell.

23. A therapeutically reprogrammed cell comprising:
a pluripotent stem cell which has been exposed to stimulatory factors which have caused said pluripotent stem cell to mature or differentiate into a more committed cell lineage.
